(19) United States
(12) Patent Application Publication
(10) Pub. No.: US 2006/0008423 A1
(43) Pub. Date: Jan. 12, 2006

(54) Dentifrice compositions and abrasive systems

(76) Inventors: Abraham Araya, Warrington (GB); Jonathan Edward Creeth, Weybridge (GB); Ian Patrick McKeown, Warrington (GB); Peter William Stanier, Warrington (GB)

Correspondence Address: GLAXOSMITHKLINE;Corporate Intellectual Property - UW2220, P.O. Box 1539, King of Prussia, PA, 19406-0939, US

(21) Appl. No.: 11/031,730

(22) Filed: Jan. 5, 2005

Related U.S. Application Data
(60) Provisional application No. 60/535,269, filed on Jan. 9, 2004.

(51) Int. Cl. A61K 8/22; A61K 8/21

(52) U.S. Cl. 424/49

(57) ABSTRACT

A dentifrice composition comprising an abrasive system comprising a combination of a crystalline aluminosilicate having an average crystallite size below 1.0 μm and a water-soluble, orally acceptable calcium sequestering agent plus an orally acceptable carrier. The crystalline aluminosilicate may have an RDA of less than 120 and a calcium binding capacity of at least 100 mg CaO per gram of anhydrous aluminosilicate. The water-soluble, orally acceptable calcium sequestering agent may be sodium tripolyphosphate.

### FIELD OF THE INVENTION

This invention relates to dentifrice compositions and abrasive systems for use in high cleaning, controlled abrasivity compositions said abrasive system comprising a combination of crystalline aluminosilicate and water-soluble calcium sequestering agent.

### BACKGROUND OF THE INVENTION

Dentifrices commonly incorporate an abrasive material for mechanical cleaning and polishing of teeth by physical abrading deposits and they may also include a chemical cleaning agent.

The abrasive material is primarily intended to effect mechanical removal of deposits from the surface of teeth, e.g. removal of pellicle film adhered to the tooth surface. Pellicle film is prone to discolouration and staining, e.g. by comestibles such as tea and coffee and by tars and particulates in exhaled cigarette smoke, resulting in an unsightly appearance of the teeth. While such mechanical removal is important for effective cleaning, it is vital that the abrasive used is not unduly harsh in order to minimise damage, e.g. scratching, to the teeth.

Synthetically produced amorphous silicas are often the favoured abrasive component in dentifrices and can be readily tailored during the production process to possess predetermined abrasive and other physical characteristics appropriate for use in dentifrices. Precipitated silicas are particularly useful as abrasive components and are generally the material of choice in dentifrice compositions.

Frequently employed chemical cleaning agents comprise water-soluble salts capable of sequestering calcium ions present in deposits on the teeth so as to counteract and reduce plaque and calculus formation. Such sequestering agents are selected in order to secure effective chemical cleaning without giving rise to undesired tooth demineralisation.

Crystalline aluminosilicates (zeolites) have been used as cleaning agents in dentifrice compositions. They possess a mechanical cleaning action (abrasivity) and are also known to bind calcium ions. Desirably, a dental cleaning agent combines relatively good cleaning with minimal abrasion of dentine. It has been found that most available zeolites are too abrasive to provide adequate cleaning without unacceptable abrasion when used in combination with silica cleaning agents.

There remains a need for formulations with improved cleaning without increased abrasivity. Surprisingly, it has now been found that the use of a combination of a specific aluminosilicate and a water-soluble, orally acceptable calcium sequestering agent can result in a dentifrice composition having good cleaning with acceptable abrasion characteristics.

### SUMMARY OF THE INVENTION

According to one aspect of the invention there is provided a dentifrice composition comprising an abrasive system comprising a combination of a crystalline aluminosilicate having an average crystallite size below 1.0 μm (typically less than 0.1 μm) and a water-soluble, orally acceptable calcium sequestering agent plus an orally acceptable carrier.

According to a second aspect of the invention there is provided a dentifrice composition comprising an abrasive system comprising a combination of a crystalline aluminosilicate having an RDA of less than 120 and a calcium binding capacity of at least 100 mg CaO per gram of anhydrous aluminosilicate and a water-soluble, orally acceptable calcium sequestering agent plus an orally acceptable carrier.

### DETAILED DESCRIPTION OF THE INVENTION

The abrasive system of the present invention may be incorporated in an orally acceptable carrier to produce a dentifrice composition. The term “orally acceptable carrier” means a suitable vehicle which can be used to apply the resulting dentifrice composition to the oral cavity in a safe and effective manner.

The water-soluble calcium sequestering agent used in the abrasive system of the invention can comprise any one or more of the following:

Water-soluble alkali metal polyphosphates (also known as condensed phosphate salts) according to formula: Mₙ₊₂[PₙO₃ₙ₊₁], where n>1, M=alkali metal, hydrogen ion or ammonium ion. Examples include: Pyrophosphates, for example alkali and mixed alkali metal salts of pyrophosphate, and pyrophosphate salts in which hydrogen ion and/or ammonium ion may partially substitute for the alkali metal ions. Examples of these are:

| | |
|---|---|
| Na₄P₂O₇ | Tetrasodium pyrophosphate |
| Na₂H₂P₂O₇ | Disodium dihydrogen pyrophosphate |
| K₄P₂O₇ | Tetrapotassium pyrophosphate |
| K₂H₂P₂O₇ | Dipotassium dihydrogen pyrophosphate |
| Na₂K₂P₂O₇ | Dipotassium disodium pyrophosphate |

Tripolyphosphates, for example alkali and mixed alkali metal salts of tripolyphosphate, and tripolyphosphate salts in which hydrogen ion and/or ammonium ion may partially substitute for the alkali metal ions. Examples are:

| | |
|---|---|
| Na₅P₃O₁₀ | Pentasodium tripolyphosphate |
| K₅P₃O₁₀ | Pentapotassium tripolyphosphate |

Higher polyphosphate salts such as sodium and potassium tetraphosphates, and hexametaphosphate salts, also known as ‘glassy phosphates’ or ‘polypyrophosphates’. Carboxylates, for example: alkali metal citrate salts, which may be partially substituted with hydrogen ion or ammonium ion, alkali metal acetate, lactate, tartrate and malate salts, which may be partially substituted with hydrogen ion or ammonium ion. Alkali metal salts of aminoacetates such as ethylenediaminetetraacetic acid (EDTA), which may be partially substituted with hydrogen ion or ammonium ion, and editronic acid.

Two or more of the above-mentioned calcium sequestering agents may be used in combination in the composition.

A preferred soluble calcium-sequestering agent is pentasodium tripolyphosphate, often referred to as sodium tripolyphosphate.

In use during tooth brushing, the calcium sequestering agent normally dissolves and so provides a cleaning effect in its dissolved state. The composition of the invention may be so formulated that the calcium-sequestering agent is in a dissolved state during use in tooth brushing, or in an aqueous vehicle.

The water soluble calcium sequestering agent, such as sodium tripolyphosphate, may be present in the range 0.1-20 percent by weight, preferably 0.25-15 percent by weight, more preferably 0.5-12 percent by weight of the dentifrice composition. By using a proportion of the calcium sequestering agent in the composition below the solubility limit thereof a gel or liquid compositions may be provided in which the calcium sequestering agent is in solution, so that the gel or liquid may include no undissolved solid particles, and may be a clear gel or liquid.

The components of the abrasive system of the invention are preferably in the dry state to ensure a free flowing powder with no microbial and preservation issues associated with filter cakes with high water content. The physical water content as measured by loss at 105° C. associated with the system and/or its individual components is preferably less than 20% of the system or individual component.

Crystalline aluminosilicates useful in this invention can be represented by the formula: M_{2/n}OAl₂O₃xSiO₂yH₂O wherein M represents a metal moiety, said metal having a valency of n, x indicates the molar ratio of silica to alumina and y indicates the ratio of molecules of water to atoms of alumina.

The structure and characteristics of many crystalline aluminosilicates (zeolites) are described in the standard work “Zeolite Molecular Sieves” by Donald W. Breck, published by Robert E. Krieger Publishing Company. Usually, the value of x in the above empirical formula is in the range 1.5 to 10. The value of y, which represents the amount of water contained in the voids of the zeolite, can vary widely. In anhydrous material y=0 and, in fully hydrated zeolites, y is typically up to 5.

Zeolites useful in this invention may be based on naturally-occurring or synthetic aluminosilicates but a preferred form of zeolite has the structure known as zeolite P. Particularly preferred forms of zeolite are those disclosed in EP-A-0 384 070, EP-A-0 565 364, EP-A-0 697 010, EP-A-0 742 780, WO-A-96/14270, WO-A-96/34828 and WO-A-97/06102, the entire contents of which are incorporated herein by this reference. The zeolite P described in EP-A-0 384 070 has the empirical formula given above in which M represents an alkali metal and x has a value up to 2.66, preferably in the range 1.8 to 2.66, and has a structure which is particularly useful in the present invention. More preferably, x has a value in the range 1.8 to 2.4. The zeolite P disclosed in the above patent literature is readily amenable to being produced with crystallite sizes well below 0.2 μm and agglomerate sizes below 2.5 μm, even when dried to a moisture content below 20% by weight. This contrasts with other zeolites which, on drying, tend to agglomerate to large weight mean particle sizes.

The average crystallite size of the crystalline aluminosilicate, measured using the test described hereinafter is preferably between 0.01 and 0.2 μm, usually between 0.02 and 0.1 μm and, more preferably between 0.02 and 0.08 μm or less.

The RDA of the crystalline aluminosilicate should be relatively low and is preferably less than 120, more preferably less than 100. Its RDA will usually be in excess of 30.

The RDA values which characterise the aluminosilicate, and other components, used in the abrasive system of this invention are measured using an aqueous slurry of the aluminosilicate, or other component, as defined in the test described hereinafter. If however the RDA were measured on the complete dentifrice composition i.e. including any optional components as defined hereinafter, the RDA values obtained may be significantly different. For example the RDA of a typical dentifrice composition incorporating an abrasive system in accordance with the present invention would be in the range 25-200, preferably 30-180, and more preferably 50-150.

Additionally, preferred aluminosilicates produce minimal scratching on dentifrice surfaces when used. Scratching can be assessed using the PAV test described hereinafter and preferred aluminosilicates have a PAV of 4 to 11, preferably 4 to 9 and more preferably 4 to 7.

The aluminosilicate preferably has a calcium binding capacity, as hereinafter defined, of at least 100 mg CaO per gram of anhydrous aluminosilicate, preferably at least 130 mg CaO per gram of anhydrous aluminosilicate and most preferably at least 150 mg CaO per gram of anhydrous aluminosilicate.

The aluminosilicate preferably has an oil absorption of at least 40 cm³/100 g and preferably in the range 40 to 100 cm³/100 g.

The aluminosilicate preferably has a weight mean particle size as measured by Malvern Mastersizer®, of at least 0.5 μm, more usually at least 1.0 μm, e.g. at least 1.8 μm. The aluminosilicate preferably has a weight mean particle size as measured by Malvern Mastersizer®, of at most 10.0 μm, more usually at most 5.0 μm e.g. at most 3.0 μm. A most preferred range for the aluminosilicate is from 2.0 to 2.5 μm.

Usually, the preferred form of zeolite P is one in which M in the above formula consists of alkali metal ions. However, suitable forms of zeolite P include those wherein a proportion of the alkali metal moieties M has been exchanged for other metal moieties, for instance as disclosed in published International Patent Application No. WO 01/94512. Partially exchanged zeolites are particularly useful when it is desired to control the pH of the abrasive system. Such pH adjustment step involves additional processing of the zeolite and associated cost. For this reason, as mentioned above, it is preferred to buffer the effect of the high pH zeolite by means of the silica content of the abrasive system and the inherent pH of the selected silica(s).

The pH of the aluminosilicate used in the abrasive system of the invention, particularly when not partially exchanged as discussed above, is usually in excess of 10. Where the aluminosilicate present in the system is one which has undergone such ion exchange, its pH will usually be no greater than 10.

The proportions of aluminosilicate and water-soluble calcium sequestering agent, e.g. alkali metal tripolyphosphate, present in the dental abrasive system of the invention can be varied in order to achieve a balance of properties suitable for the dentifrice composition in which it is used. Generally, the proportion of aluminosilicate to water-soluble agent by weight is in the range 400:1 to 1:2. Preferably, the ratio is in the range 80:1 to 2:3, most preferably in the range 30:1 to 1:1 aluminosilicate to water-soluble agent by weight.

When a dentifrice composition is prepared using the abrasive system of this invention, the components of the system (including any additional components as referred to below) may be mixed prior to combining the subsequent mixture with the other components of the dentifrice composition or may be separately added to the other components of the dentifrice composition. In each instance, the components (including any additional components of the system as referred to below) or mixture thereof will, at least prior to combining the same with other components of the dentifrice composition, usually be in the form of a substantially dry free flowing particulate material.

Additional components may also be present in the dental abrasive system of the invention. One such component is a moderately abrasive amorphous silica, which has a low to medium RDA within the range 30 to 150. Typically its RDA is at least 40, more usually at least 50. Typically its RDA is no greater than 130, e.g. 110. It typically has an oil absorption of 60 to 140 cm³/100 g, preferably 80 to 120 cm³/100 g. It typically has a weight mean particle size in the range 5 to 15 μm, preferably 6 to 12 μm, the size being measured by a Malvern Mastersizer®, as described hereinafter.

Another such component is an abrasive amorphous silica, which is capable of acting as a booster to the cleaning ability of the system. Preferred silicas suitable as boosters have an RDA of 100 to 300, preferably 100 to 220. The silica preferably has an oil absorption of 40 to 150 cm³/100 g, and more preferably 40 to 100 cm³/100 g. The weight mean particle size of the silica is preferably in the range 3 to 15 μm. More preferably, the silica has a weight mean particle size in the range 3 to 6 μm. Preferably, the amorphous silica or silicas employed is/are precipitated silica(s).

A further additional component can be a different crystalline aluminosilicate, e.g. an A, X or Y type zeolite, which acts as a cleaning booster (hereinafter referred to as “booster zeolite”). When present, the amount of booster zeolite present will usually be less than that of the zeolite referred to hereinbefore (the “principal” zeolite). This booster zeolite preferably has an RDA in the range 100 to 300 and more preferably in the range 100 to 250. The PAV of the booster zeolite is preferably in the range 9 to 25 and more preferably in the range 9 to 20. The values for both the RDA and the PAV of the booster zeolite will be greater than those for the principal zeolite. The preferred oil absorption of the booster zeolite is in the range 30 to 100, more preferably in the range 30 to 50 cm³/100 g. The weight mean particle size of the booster zeolite is preferably in the range 2.0 to 5.0 μm. The booster zeolite preferably has an average crystallite size above 0.2 μm and most preferably above 1.0 μm.

The proportions of crystalline aluminosilicate and one or more additional particulate materials selected from moderately abrasive silica, booster silica or booster zeolite present in the dental abrasive system of the invention can be varied to provide optimum cleaning with controlled abrasion. Generally, the proportion of crystalline aluminosilicate to such additional particulate materials, usually booster particles, by weight is in the range 40:1 to 1:1. Preferably, the ratio is in the range 9:1 to 3:2. The term “booster particles”, as used herein, refers to booster silica, booster zeolite or a combination of booster silica and booster zeolite.

A dentifrice composition containing the abrasive system according to the present invention may also include a fluoride ion source as protection against demineralisation by bacteria (caries) and/or acidic components of the diet (erosion). The fluoride ion source may be provided by any of the compounds conventionally used in toothpastes for these purposes, e.g. sodium fluoride, alkali metal monofluorophosphate, stannous fluoride and the like, with an alkali metal monofluorophosphate such as sodium monofluorophosphate being preferred. The fluoride ion source serves in a known manner for caries protection. Preferably, the fluoride ion source will be used in an amount to provide a safe yet effective amount to provide an anti-caries and anti-erosion benefit such as an amount sufficient to provide from about 25 ppm to about 3500 ppm, preferably about 1100 ppm, as fluoride ion. For example the formulation may contain 0.1-0.5 wt % of an alkali metal fluoride such as sodium fluoride.

Preferably the pH of the dentifrice composition incorporating an abrasive system of the present invention is from about 6 to 10.5, more preferably from about 7 to about 9.5.

Typically the composition may contain sodium hydroxide, e.g. up to 1.0 wt. % to provide a suitable pH.

In compositions containing an abrasive system in accordance with the present invention which are usable in the manner of conventional toothpastes, i.e. which can be extruded onto a toothbrush, the orally acceptable vehicle may be of a generally conventional composition e.g. comprising a thickening agent, a binding agent and a humectant. Preferred binding agents include for example natural and synthetic gums such as xanthan gums, carageenans, alginates, cellulose ethers and esters. Preferred humectants include glycerin, sorbitol, propylene glycol and polyethylene glycol. A preferred humectant system consists of glycerin, sorbitol and polyethylene glycol.

In addition, the orally acceptable vehicle may optionally comprise one or more surfactants, sweetening agent, flavouring agent, anticaries agent (in addition to the fluoride ion source), anti-plaque agent, anti-bacterial agent such as triclosan or cetyl pyridinium chloride, tooth desensitizing agent such as potassium or strontium salts such as potassium nitrate or strontium chloride, colouring agents and pigment. Useful surfactants include the water-soluble salts of alkyl sulphates having from 10 to 18 carbon atoms in the alkyl moiety, such as sodium lauryl sulphate, but other anionic surfactants as well as non-ionic, zwitterionic and cationic surfactants may also be used.

If an aqueous orally acceptable vehicle is employed, the dentifrice composition suitably contains from about 10 to about 80 wt % humectant such as sorbitol, glycerin, polyethylene glycol or xylitol; from about 0.25 to about 5 wt % detergent; from 0 to about 2 wt % sweetener; from 0 to about 2 wt % flavouring agents; together with water and an effective amount of binding and thickening agents, such as from about 0.1 to about 15 wt %, to provide the toothpaste of the invention with the desired stability and flow characteristics.

As previously stated, the abrasive systems of the invention are capable of providing dentifrice compositions with good cleaning and within the abrasion limits generally considered as acceptable. The cleaning ability of a composition can be assessed by the test known as the NESR test (see Creeth J E, Wicks M A, Whitworth D, McConville P S.

Improved in vitro model for developing toothpastes with optimised whitening performance. J. Dent. Res. 81 poster 652, 2002).

The tests used to characterise the components of the abrasive system of this invention are as follows.

Radioactive Dentine Abrasion Test (RDA)

The procedure follows the method for assessment of dentifrice abrasivity recommended by the American Dental Association (Journal of Dental Research 55(4) 563, 1976). In this procedure, extracted human teeth are irradiated with a neutron flux and subjected to a standard brushing regime. The radioactive phosphorus 32 removed from the dentin in the roots is used as the index of the abrasion of the dentifrice tested. A reference slurry containing 10 g of calcium pyrophosphate in 50 cm³ of 0.5% aqueous solution of sodium carboxymethyl cellulose is also measured and the RDA of this mixture is arbitrarily taken as 100. In order to measure a powder RDA for the crystalline aluminosilicate or silica a suspension of 10.0 g of the silica or aluminosilicate in 50 cm³ of 0.5% aqueous solution of sodium carboxymethyl cellulose is prepared and the suspension is submitted to the same brushing regime. In order to measure an RDA value for a dentifrice composition containing an abrasive system of the invention a test slurry is prepared from 25 g dentifrice composition and 40 cm³ of water and this slurry is submitted to the same brushing regime.

Plastics Abrasion Value (PAV)

This test is based upon a toothbrush head brushing a Perspex® plate in contact with a suspension of the aluminosilicate in a sorbitol/glycerol mixture. Perspex® has a similar hardness to dentine. Therefore, a substance which produces scratches on Perspex® is likely to produce a similar amount of scratching on dentine. Normally the slurry concentration is as follows:

| | |
|---|---|
| Aluminosilicate | 2.5 g |
| Glycerol | 10.0 g |
| Sorbitol Syrup* | 23.0 g |

*Syrup contains 70% sorbitol/30% water

All components are weighed into a beaker and dispersed for 2 minutes at 1500 rpm using a simple stirrer. A 110 mm×55 mm×3 mm sheet of standard PERSPEX clear cast acrylic sheet, grade 000, manufactured by Lucite International UK Ltd, PO Box 34, Darwen, Lancashire, UK, is used for the test.

The test is carried out using a modified Wet Scrub Abrasion Tester produced by Sheen Instruments. The modification is to change the holder so that a toothbrush can be used in place of a paintbrush. In addition, a weight of 400 g is attached to the brush assembly, which weighs 145 g, to force the brush onto the PERSPEX sheet. The toothbrush has a multi-tufted, flat trim nylon head with round ended filaments and medium texture, for example, the well-known Professional Mentadent P gum health design, or an equivalent toothbrush.

A galvanometer is calibrated using a 45° Plaspec gloss head detector and a standard (50% gloss) reflecting plate. The galvanometer reading is adjusted to a value of 50 under these conditions. The reading of the fresh PERSPEX sheet is then carried out using the same reflectance arrangement.

The fresh piece of PERSPEX sheet is then fitted into a holder. 2 cm³ of the dispersed aluminosilicate, sufficient to lubricate fully the brushing stroke, is placed on the sheet and the brush head is lowered onto the sheet. The machine is switched on and the sheet is subjected to 300 strokes of the weighted brush head. The sheet is removed from the holder and all the suspension is washed off. It is then dried and its gloss value is determined again. The abrasion value is the difference between the unabraded gloss value and the gloss value after abrasion. This test procedure, when applied to known abrasives, gave the following typical values.

| | PAV |
|---|---|
| Calcium carbonate (15 μm) | 32 |
| Silica xerogel (10 μm) prepared according to GB 1 262 | 25 |
| 292 | |
| Alumina trihydrate (Gibbsite) (15 μm) | 16 |
| Calcium pyrophosphate (10 μm) | 14 |
| Dicalcium phosphate dihydrate (15 μm) | 7 |

Oil Absorption

The oil absorption is determined by the ASTM spatula rub-out method (American Society of Test Material Standards D 281). The test is based on the principle of mixing linseed oil with the silica or aluminosilicate by rubbing with a spatula on a smooth surface until a stiff putty-like paste is formed which will not break or separate when it is cut with a spatula. The oil absorption is then calculated from the volume of oil (V cm³) used to achieve this condition and the weight, W, in grams, of silica or aluminosilicate by means of the equation:
Oil absorption=(V×100)/W, i.e. expressed in terms of cm³ oil/100 g silica or aluminosilicate.
Weight Mean Particle Size by Malvern Mastersizer®

The weight mean particle size of the silica or aluminosilicate is determined using a Malvern Mastersizer® model S, with a 300 RF lens and MS 17 sample presentation unit. This instrument, made by Malvern Instruments, Malvern, Worcestershire, uses the principle of Fraunhofer diffraction, utilising a low power He/Ne laser. Before measurement, the sample is dispersed ultrasonically in water for 5 minutes (in the case of silica) and 30 seconds (in the case of aluminosilicate) to form an aqueous suspension. The Malvern Mastersizer® measures the weight particle size distribution of the silica or aluminosilicate. The weight mean particle size (d₅₀) or 50 percentile and the percentage of material below any specified size are easily obtained from the data generated by the instrument.

Average Crystallite Size of Aluminosilicate

The average crystallite size is determined from photographs made in a scanning electron microscope. The crystalline aluminosilicate is dried to a water content of about 1 to 3 weight percent and the agglomerates are broken up with a pestle and mortar. From the photographs, a sufficient number of crystals, e.g. 100, is counted and their size measured to determine a statistically significant average (arithmetical mean) size.

Effective Calcium Binding Capacity of Aluminosilicate

The aluminosilicate is first equilibrated to constant weight over saturated sodium chloride solution and the water content is measured. An amount is dispersed in 1 cm³ water in an amount corresponding to 1 g dm⁻³ (dry weight) and the resulting dispersion is injected into a stirred solution of total volume 54.923 cm³, consisting of 0.01M NaCl solution (50 cm³) and 0.05M CaCl₂ (3.923 cm³). This corresponds to a concentration of 200 mg of CaO per dm³, i.e. just greater than the theoretical maximum amount (197 mg) that can be taken up by an aluminosilicate of Si:Al ratio 1.00. The dispersion is vigorously stirred at a temperature of 25° C. for 15 minutes, after which time the Ca²⁺ ion concentration is determined using a calcium electrode. The Ca²⁺ ion concentration measured is subtracted from the initial concentration to give the effective calcium binding capacity of the aluminosilicate sample.

Natural Extrinsic Stain Removal Test.

The natural extrinsic stain removal (NESR) test is an in vitro brushing method that uses bovine enamel as the stained substrate. Bovine teeth are extracted from jaws obtained from approved sources, depulped, disinfected in a solution of thymol and accepted or rejected for mounting based upon a visual assessment of the quantity of stain present on the surface of the tooth. The stained bovine enamel specimens are then mounted into Ecotrin 30 cc bottle caps (3.5 cm diameter×1.5 cm depth) using acrylic powder and liquid. A weartesting machine is used consisting of 28 stations into which mounted teeth and test solutions/slurries are placed. Each station (or tray) is associated with a dedicated toothbrush (in particular, an “Oral B” 40 flat trim toothbrush or equivalent thereof) supported above the tray on a hinged metal arm. Mounted bovine specimens are sited within each tray such that, when the metal arm is placed in the “down” position and the weartester activated, a continuous brushing motion over the surface of each tooth occurs at a fixed rate in a direction parallel to the surface of the bovine enamel specimen. The force applied per stroke is targeted at approximately 100 g, and a stroke rate of 100 strokes per minute. Each mounted specimen is marked with 5 notches in permanent ink, positioned in an equidistant manner around the circumference of the Ecotrin cap. Gross amounts of extrinsic stain are removed from bovine enamel specimens by performing a 10 minute “pre-brush” using a 1:3 slurry of Macleans Milk Teeth toothpaste in de-ionised water, filled until the top of the tooth is just covered with test slurry. When the 10-minute pre-brush block is completed, each bovine enamel specimen is rinsed with de-ionised water and allowed to dry overnight. The level of stain is assessed via the L value of the CIELAB L*a*b* scale measured using a Huntercolour L*a*b spectrocolourimeter (Model LS6100). Five readings per tooth are recorded and the mean determined. Bovine enamel specimens with L-values ranging between 50 and 85 are selected. The bovine enamel specimens were then ranked by L* value, and randomised across test cells to minimise experimental bias.

The test is run as a two-product head-to-head protocol, i.e. Test Product 1 (X) vs. Test Product 2 (Y). The teeth are randomly divided into two sets. In the first treatment phase (T1), one set of teeth is brushed with a 1+1 w/w slurry of X in de-ionised water for 30 minutes and the second set is brushed with Y under the same conditions. The teeth are washed and dried overnight as above. The L* value is recorded and then the teeth undergo a second treatment phase (T2). In this phase (T2), the set previously treated with X is brushed with a 1:1 slurry of Y in de-ionised water, and the set treated with Y is likewise brushed with X. The teeth are washed, dried and the L* value measured as previously.

The relative stain removal efficacy of X versus Y is determined by comparing, for each treatment sequence, the percentage of stain removed during the first treatment phase as a proportion of the total amount of stain removed by both treatment phases. That is, whether:
[ΔL*(T1[X])/ΔL*(T1[X])+ΔL*(T2[Y])]×100
is statistically significantly different from (p<0.05):
[ΔL*(T1[Y])/ΔL*(T1[Y])+ΔL*(T2[X])]×100

Values are constrained to be within the range 0-100%.

Dentifrice Formulation 1

| INGREDIENT | % by wt. |
|---|---|
| Sorbitol, 70% Soln. | 26.00 |
| Glycerol | 10.00 |
| Polyethylene glycol | 3.00 |
| Crystalline aluminosilicate | A |
| Booster Particle | B |
| Water soluble calcium sequestering | C |
| agent | |
| Silica thickener | D |
| Sodium lauryl sulphate | 1.15 |
| Titanium Dioxide | 1.45 |
| Xanthan gum | 0.7 |
| Saccharin | 0.23 |
| Flavor | 1 |
| NaF | 0.24 |
| De-ionized water | to 100 |
| Total | 100.00 |

The quantities A, B and C are determined by the abrasive system under test (see Examples below). The quantity of thickening silica (“D”) is adjusted to ensure that the cohesion of the paste, as measured by the toothpaste cohesion test defined hereinafter, is in the range 150 to 430 g.

Toothpaste Cohesion

The cohesion of a toothpaste is a good measure of the “stand-up” properties of the ribbon when it has been extruded from a toothpaste tube onto a toothbrush. Higher cohesion values indicate firmer toothpaste ribbons, whereas low cohesion numbers are obtained from low viscosity, poorly structured toothpastes, which quickly sag into the bristles of the brush. It is generally required that a dentifrice has a cohesion within the range of 150-430 g to provide a good quality, extrudable ribbon, which does not sag and is not too firm.

The basic principle of the test is to measure the weight in grams required to pull two parallel plates apart, which have a specific layer of toothpaste sandwiched between them. The purpose built equipment consists of:

1) A spring balance in which the spring can be extended from 0-430 g in 100 mm of length. The spring has a calibration scale of zero to 430 g in 10 g intervals and can be adjusted to zero at the start of the test.

2) A motor driven ratchet, which is attached to the bottom plate and can be used to apply a constant, uniform, smooth vertical pull on the bottom plate of 5 cm per minute.

3) An upper polished chrome circular plate of 64 mm diameter, which has a hook on the upper side that can be attached to the spring balance. The polished plate has three small identical spacer pieces of polished chrome on the underside of the plate, as an integral part of the plate. These protrude to a depth of 4 mm, which determines the toothpaste film thickness when the equipment is assembled to carry out the test.

4) A lower polished chrome circular plate of 76 mm diameter, which is attached underneath to a motor driven ratchet. Two short pegs are located on the top of the plate so that the top plate can be positioned on the bottom plate concentrically from the centres.

5) A metal framework which allows the top plate to be situated concentrically above the bottom plate and the bottom plate to be adjusted so that the plate is approximately horizontal (achieved through the use of levelling feet on the base of the equipment).

15-20 g of toothpaste is evenly distributed onto the underside of the upper plate and the plate is carefully positioned onto the top of the bottom plate, using the two short pegs to locate the edge of the top plate. The top plate is firmly pressed down onto the bottom plate, until all three spacers have made contact with the bottom plate. Excess toothpaste, which has been squeezed out from between the two plates is then removed with a spatula, such that no toothpaste extends beyond the diameter of the top plate. The upper plate is then connected to the spring balance and the scale set to zero grams. The equipment is then switched on to allow the motor driven ratchet to lower the bottom plate. The spring is gradually extended and the highest observed weight is noted, as the two parallel plates sandwiched with toothpaste are eventually pulled apart. This is the toothpaste cohesion recorded in grams.

pH

This measurement is carried out on a 5 weight percent suspension of the silica or aluminosilicate in boiled demineralised water (CO₂ free).

Ignition Loss at 1000° C.

Ignition loss is determined by the loss in weight of a silica when ignited in a furnace at 1000° C. to constant weight.

Moisture Loss at 105° C.

Moisture loss is determined by the loss in weight of a silica when heated in an oven at 105° C. to constant weight.

The invention is illustrated by the following non-limiting examples.

### EXAMPLES

In order to demonstrate the use of the invention, the aforementioned Dentifrice Formulation 1 was used as a base formulation in which particle components A, B, C and D were varied according to the following examples and reference example:

### Example 1

Dentifrice formulation 1 was produced using 30% by weight Doucil A24 Zeolite, as the crystalline aluminosilicate (A), 10% by weight STPP-sodium tripolyphosphate-(C), and 5% by weight thickening silica (D) having a pH of 6.4. The properties of Doucil A24 Zeolite are given in Table 1. The toothpaste had an RDA of 104 and the cleaning data is given in Table 3.

### Example 2

Dentifrice formulation 1 was produced using 14% by weight Doucil A24 Zeolite as the crystalline aluminosilicate (A), 10% by weight sodium tripolyphosphate (C), 5% by weight thickening silica (D) having a pH of 6.4 and 4.2% by weight Sorbosil AC43 (as booster silica particles, B). The properties of the cleansing particles used are given in Tables 1 and 2. The toothpaste had an RDA of 114 and the cleaning data is given in Table 3.

TABLE 1
Zeolite Powder Properties

| | | | Oil | Weight | | | Calcium |
|---|---|---|---|---|---|---|---|
| | | | absorption | mean | Average | | binding |
| | Powder | | (cm³/ | particle size | Crystallite | | capacity mg |
| Particle type | RDA | PAV | 100 g) | (μm) | size (μm) | pH | CaO/g |
| Doucil A24 | 82 | 8 | 58 | 2.25 | 0.06 | 11.4 | 160 |
| Zeolite | | | | | | | |

Doucil A24 Zeolite is a crystalline aluminosilicate available from INEOS Silicas Limited, Warrington, UK.

TABLE 2
Abrasive Silica Powder Properties

| | | | | Ignition | |
|---|---|---|---|---|---|
| | | | Weight mean | Loss at | |
| | Powder | Oil absorption | particle size | 1000° C. | |
| Particle type | RDA | (cm³/100 g) | (μm) | (%) | pH |
| Sorbosil AC43 | 160 | 75 | 3.5 | 11.0 | 5.5 |

Sorbosil AC43 is a toothpaste cleaning booster silica available from INEOS Silicas Limited, Warrington, UK.

For comparison with Example 1, Dental Formulation 1 was produced using a standard silica abrasive (Control 1). In the comparative formulation, A and B were 0%, C was 10% and D was 6.5%. 14% by weight of silica with powder RDA=85 and oil absorption=90 was used as the standard silica abrasive. The RDA of this formulation is about 85. The cleaning data are presented in Table 3. The data show a substantial increase in cleaning efficacy for a modest increase in abrasivity.

For comparison with Example 2, Dental Formulation 1 was also produced as a second control using a different standard silica abrasive (Control 2). In this comparative formulation, A and B were 0%, C was 10% and D was 5.5%. 16% by weight of abrasive silica with powder RDA=95 and oil absorption=107 was used as the standard silica abrasive. The RDA of this formulation is about 130. The cleaning data are presented in Table 3. The data show that the aluminosilicate formulation gives equivalent cleaning efficacy but lower abrasivity.

TABLE 3
Toothpaste cleaning properties

| | % stain removed | Significance |
|---|---|---|
| Treatment | by 1^{st} treatment | of difference |
| Example 1: 30% Zeolite + STPP | 68.27 | p = 0.0224 |
| Control 1 | 44.64 | |
| Example 2: 14% Zeolite + | 77.74 | P = 0.75 |
| 10% STPP + 4.2% AC43 | | |
| Control 2 | 76.63 | |

What is claimed is:

1. An abrasive system forming part of a dentifrice composition comprising a combination of a crystalline aluminosilicate having an average crystallite size below 1.0 μm and a water-soluble, orally acceptable calcium sequestering agent.

2. An abrasive system forming part of a dentifrice composition comprising a combination of a crystalline aluminosilicate having an RDA of less than 120 and a calcium binding capacity of at least 100 mg CaO per gram of anhydrous aluminosilicate and a water-soluble, orally acceptable calcium sequestering agent.

3. An abrasive system as claimed in claim 1 in which the components of the system are in the dry state to ensure a free flowing powder.

4. An abrasive system as claimed in claim 1 in which the aluminosilicate comprises a zeolite having the formula;
M_{2/n}OAl₂O₃xSiO₂yH₂O

5. An abrasive system as claimed in claim 4 in which the zeolite is a P zeolite.

6. An abrasive system as claimed in claim 4 in which x has a value up to 2.66.

7. An abrasive system as claimed in claim 4 in which x has a value in the range 1.8 to 2.66.

8. An abrasive system as claimed in claim 4 in which x has a value in the range 1.8 to 2.4.

9. An abrasive system as claimed in claim 1 in which the aluminosilicate has a moisture content below 20% by weight.

10. An abrasive system as claimed in claim 1 in which the average crystallite size of the crystalline aluminosilicate is between 0.01 and 0.2 μm.

11. An abrasive system as claimed in claim 1 in which the average crystallite size of the crystalline aluminosilicate is less than 0.1 μm.

12. An abrasive system as claimed in claim 1 in which the average crystallite size of the crystalline aluminosilicate is up to 0.08 μm.

13. An abrasive system as claimed in claim 1 in which the RDA of the crystalline aluminosilicate is not greater than 120.

14. An abrasive system as claimed in claim 1 in which the aluminosilicate has a PAV of up to 11, preferably up to 9.

15. An abrasive system as claimed in claim 1 in which the aluminosilicate has a calcium binding capacity of at least 130 mg CaO per gram of anhydrous aluminosilicate.

16. An abrasive system as claimed in claim 1 in which the aluminosilicate has an oil absorption of at least 40 cm³/100 g.

17. An abrasive system as claimed in claim 1 in which the aluminosilicate has a weight mean particle size of at least 0.5 μm.

18. An abrasive system as claimed in claim 1 in which the aluminosilicate has a weight mean particle size of at most 10.0 μm.

19. An abrasive system as claimed in claim 1 in which the aluminosilicate has a weight mean particle size in range 2.0 to 2.5 μm.

20. An abrasive system as claimed in claim 1 in which the crystalline aluminosilicate is one which has been ion exchanged in order to reduce its pH.

21. An abrasive system as claimed in claim 1 in which the crystalline aluminosilicate is derived from a zeolite P with the formula M_{2/n}OAl₂O₃xSiO₂yH₂O where M is an alkali metal and in which at least a proportion of the alkali metal M has been exchanged for one or more other metal moieties.

22. An abrasive system as claimed in claim 1 in which the sequestering agent is selected one or more of the group comprising alkali metal polyphosphates, carboxylates and alkali metal salts of aminoacetates.

23. An abrasive system as claimed in claim 1 in which the calcium-sequestering agent is sodium tripolyphosphate.

24. An abrasive system as claimed in claim 1 further comprising a moderately abrasive amorphous silica having an RDA of at least 30.

25. An abrasive system as claimed in claim 24 in which said moderately abrasive silica has an RDA within the range 30 to 150.

26. An abrasive system as claimed in claim 24 in which said moderately abrasive silica has an oil absorption of 60 to 140 cm³/100 g.

27. An abrasive system as claimed in claim 24 in which said moderately abrasive silica has a weight mean particle size in the range 5 to 15 μm.

28. An abrasive system as claimed in claim 24 further comprising a relatively highly abrasive amorphous silica.

29. An abrasive system as claimed in claim 1 further comprising a relatively highly abrasive amorphous silica (booster silica).

30. An abrasive system as claimed in claim 29 in which the booster silica has an RDA of 100 to 300.

31. An abrasive system as claimed in claim 29 in which the booster silica has an oil absorption of 40 to 150 cm³/100 g.

32. An abrasive system as claimed in claim 29 in which the booster silica has weight mean particle size in the range 3 to 15 μm.

33. An abrasive system as claimed in claim 1 further comprising a different crystalline aluminosilicate which acts as a cleaning booster.

34. An abrasive system as claimed in claim 33 in which said cleaning booster has an RDA in the range 100 to 300.

35. An abrasive system as claimed in claim 33 in which said cleaning booster has a PAV in the range 9 to 25.

36. An abrasive system as claimed in claim 33 in which said cleaning booster has an oil absorption in the range 30 to 100 cm³/100 g.

37. An abrasive system as claimed in claim 33 in which said cleaning booster has a weight mean particle size of the booster zeolite in the range 2.0 to 5.0 μm.

38. An abrasive system as claimed in claim 33 in which said cleaning booster has an average crystallite size above 0.2 μm.

39. A dentifrice composition comprising an abrasive system comprising a combination of a crystalline aluminosilicate having an average crystallite size below 1.0 μm and a water-soluble, orally acceptable calcium sequestering agent plus an orally acceptable carrier.

40. A dentifrice composition comprising an abrasive system comprising a combination of a crystalline aluminosilicate having an RDA of less than 120 and a calcium binding capacity of at least 100 mg CaO per gram of anhydrous aluminosilicate and a water-soluble, orally acceptable calcium sequestering agent plus an orally acceptable carrier.

41. A dentifrice composition as claimed in claim 1 in which the sequestering agent is selected from one or more of the group comprising alkali metal polyphosphates, carboxylates and alkali metal salts of aminoacetates.

42. A dentifrice composition as claimed in claim 1 in which the calcium-sequestering agent is sodium tripolyphosphate.

43. A dentifrice composition as claimed in claim 1 in which the calcium sequestering agent is present in the range 0.1-20% by weight.

44. A dentifrice composition as claimed in claim 1 in which the aluminosilicate comprises a zeolite having the formula; M_{2/n}OAl₂O₃xSiO₂yH₂O

45. A dentifrice composition as claimed in claim 44 in which the zeolite is a P zeolite.

46. A dentifrice composition as claimed in claim 1 in which the RDA of a complete formulation is 25-200.

47. A dentifrice composition as claimed in claim 1 wherein the pH is from 6 to 10.5.

48. A dentifrice composition as claimed in claim 1 further comprising a fluoride source.
